(19) United States
(12) Patent Application Publication
(10) Pub. No.: US 2012/0271102 A1
(43) Pub. Date: Oct. 25, 2012

(54) STEREOSCOPIC ENDOSCOPE APPARATUS

(75) Inventor: Akihiro Katayama, Zama-shi (JP)

(73) Assignee: CANON KABUSHIKI KAISHA, Tokyo (JP)

(21) Appl. No.: 13/412,701

(22) Filed: Mar. 6, 2012

(30) Foreign Application Priority Data
Apr. 21, 2011 (JP) 2011-095206

(51) Int. Cl. A61B 1/04; A61B 1/018

(52) U.S. Cl. 600/104

(57) ABSTRACT

To provide a stereoscopic endoscope apparatus capable of reducing the visual interference caused when a treatment instrument approaches an imaging unit. Provided is a stereoscopic endoscope apparatus that includes a plurality of imaging units and a channel having an operable treatment instrument inserted therethrough and stereoscopically displays an image obtained from the plurality of imaging units, the stereoscopic endoscope including: a front end detecting unit that detects a front end of the treatment instrument within the image acquired by the imaging units; an area determining unit that determines an area to be displayed as a two-dimensional image; and a two-dimensional image processing unit that two-dimensionally images the area to be displayed as a two-dimensional image, in which the area to be displayed as a two-dimensional image is an area where the treatment instrument is seen.

### BACKGROUND OF THE INVENTION

1. Field of the Invention

The present invention relates to a stereoscopic endoscope apparatus, and particularly, to an image process for satisfactorily observing a subject at a comparatively close distance using a plurality of imaging units installed in a front end of the stereoscopic endoscope apparatus.

2. Description of the Related Art

As illustrated in FIG. 8, a conventional stereoscopic endoscope is provided with stereo cameras (101R and 101L), a channel 102 through which a treatment instrument is inserted, illumination lamps 103 and 104, which are arranged at a front end of an endoscope 100 to be inserted into a subject. A doctor conducts a surgical operation using the treatment instrument and the like while observing a stereoscopic image obtained by three-dimensionally displaying images acquired from the stereo cameras (101R and 101L).

In such a stereoscopic endoscope, since the convergence angle which is determined by the stereo cameras is too large with respect to the treatment instrument that closely approaches the stereo cameras, stereoscopic displaying cannot be easily performed. For example, with regard to the images which are acquired by the stereo cameras 101R and 101L in a state where the treatment instrument is delivered from the channel 102, the image of the stereo camera 101L is acquired as illustrated in FIG. 9A and the image of the stereo camera 101R is acquired as illustrated in FIG. 9B, and the treatment instruments (which are depicted as dotted bars in the respect drawings) are appeared with different angles in the images.

In this state, the treatment instruments within the images acquired from the respective cameras overlap each other in a deviated state as illustrated in FIG. 9C. As can be seen in the figure, in the vicinity of the corner of the image where the delivered treatment instrument is appeared, the disparity of the stereoscopic image is too large, so that the stereoscopic image may not be displayed.

In order to solve this problem, Japanese Patent Application Laid-Open No. 2004-65804 discloses a method of two-dimensionally displaying a predetermined area or a method of generating a mask image and overlapping the mask image with an original image.

### SUMMARY OF THE INVENTION

However, in the related art which is disclosed in Japanese Patent Application Laid-Open No. 2004-65804, an area which is not easily displayed as a stereoscopic image is obtained in advance and only a single-eye image for that area is displayed or the area is overlapped with the mask image. For this reason, there is a problem that other areas are also displayed as a two-dimensional image or displayed with a mask, even when the treatment instrument is slightly delivered from the channel. The invention is made in view of the above-described problems, and it is an object of the invention to provide a structure capable of ensuring a stereoscopically displayed area by detecting a front end of a treatment instrument and with minimized amount of processes.

In order to attain the above-described object, the apparatus according to an aspect of the invention includes the following configuration. That is, a first aspect of the invention relates to a stereoscopic endoscope apparatus capable of operating a treatment instrument through a channel and stereoscopically displaying an image acquired by a plurality of imaging units, the stereoscopic endoscope apparatus including: a front end detecting unit that detects a front end of the treatment instrument in at least one arbitrary image from the plurality of images acquired by the plurality of imaging units; an area determining unit that determines an area which is distant from the front end of the treatment instrument by a predetermined distance and is to be displayed as a two-dimensional image; and a two-dimensional image processing unit that two-dimensionally displays the area in an image used for the stereoscopic display in the plurality of images acquired by the plurality of imaging units.

Further, a second aspect of the invention relates to a stereoscopic endoscope apparatus capable of operating a treatment instrument through a channel and stereoscopically displaying an image acquired by a plurality of imaging units, the stereoscopic endoscope apparatus including: a front end detecting unit that detects a front end of the treatment instrument in at least one arbitrary image from the plurality of images acquired by the plurality of imaging units; a deformation area determining unit that determines an area where an image is deformed within an image distant from the front end of the treatment instrument by a predetermined distance; and a deformation unit that deforms the deformation area determined in the images used for the stereoscopic display in the plurality of images acquired by the plurality of imaging units in a shape in which the disparity of the stereoscopic image decreases.

According to the invention, since the visual interference may be reduced by processing only an area which is essentially required in a stereoscopic observation area, a more easily operable stereoscopic endoscope can be provided.

Further features of the present invention will become apparent from the following description of exemplary embodiments with reference to the attached drawings.

### BRIEF DESCRIPTION OF THE DRAWINGS

FIG. 1 is a diagram illustrating a functional configuration of a process system according to a first embodiment.

FIGS. 2A, 2B, and 2C are diagrams illustrating a front end detecting unit.

FIGS. 3A, 3B, and 3C are diagrams illustrating an image processing unit.

FIGS. 4A and 4B are diagrams illustrating a second embodiment.

FIG. 5 is a diagram illustrating apparatus configurations of the first embodiment and the second embodiment.

FIG. 6 is a diagram illustrating a flow of a process of the first embodiment.

FIG. 7 is a diagram illustrating a flow of a process of the second embodiment.

FIG. 8 is a diagram illustrating an example of (only a front end of) an existing stereoscopic endoscope.

FIGS. 9A, 9B, and 9C are diagrams illustrating an example of an image that is acquired by the existing stereoscopic endoscope.

### DESCRIPTION OF THE EMBODIMENTS

Preferred embodiments of the present invention will now be described in detail in accordance with the accompanying drawings.

Hereinafter, exemplary embodiments of a processing apparatus and a processing method according to the invention will be described in detail according to the accompanying drawings. However, the scope of the invention is not limited to the example illustrated in the drawings.

### First Embodiment

A stereoscopic endoscope apparatus according to the embodiment reduces the visual interference by detecting a front end of a treatment instrument which is operable through a channel and displaying a portion of the treatment instrument other than a predetermined area of the front end as a two-dimensional image.

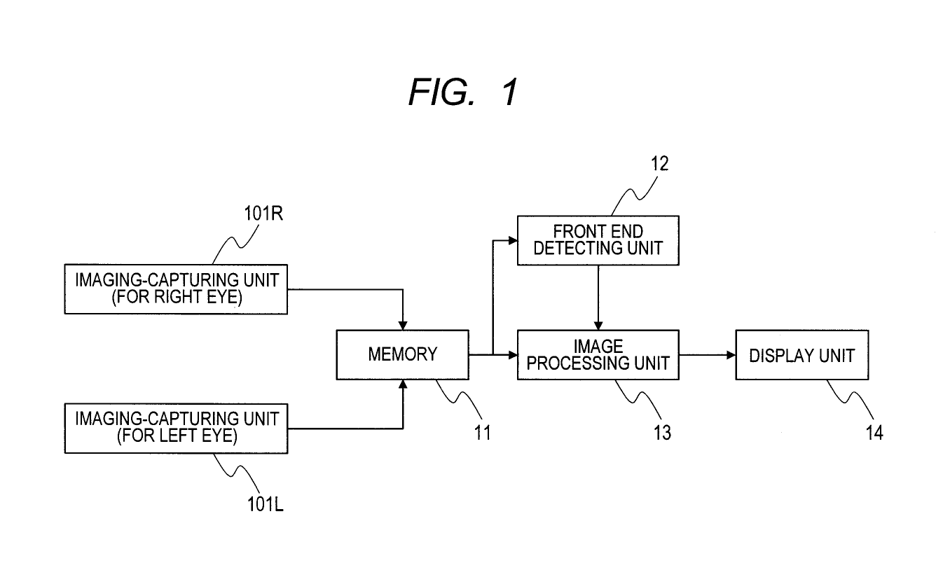

FIG. 1 is a functional block diagram of the embodiment. In the drawing, imaging units 101R and 101L are provided for left and right eyes. A memory 11 for storing the images which are acquired by the imaging unit 101R and 101L is provided. A front end detecting unit 12 detects the front end of the treatment instrument. An image processing unit 13 creates a two-dimensional image of the treatment instrument other than a portion up to a predetermined distance from the detected front end. As described below, the image processing unit includes an area determining unit and a two-dimensional image processing unit. The front end detecting unit 12 detects the front end of the treatment instrument from the images which are stored in the memory 11, and transmits information thereof to the image processing unit 13. In the image processing unit 13, the area determining unit determines, based on the information, an area where the image is to be displayed as a two-dimensional image, and the two-dimensional image processing unit performs the two-dimensional image process. The display unit 14 displays the as a stereoscopic image.

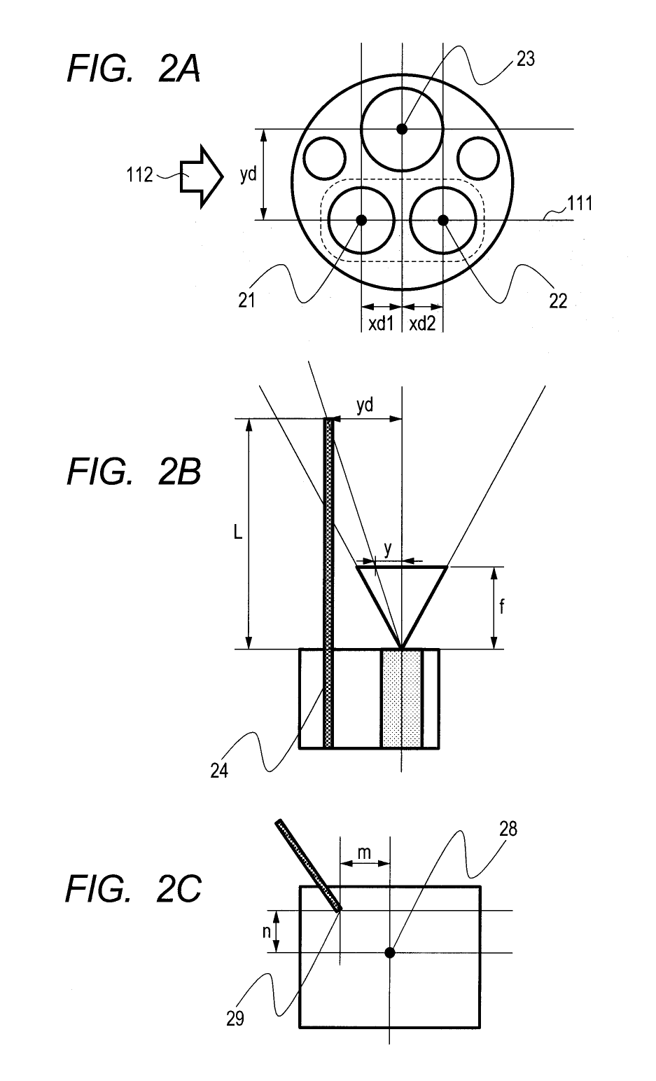

Next, the process of the front end detecting unit will be described by referring to FIGS. 2A, 2B, and 2C. FIG. 2A illustrates the imaging unit of the stereoscopic endoscope. The shortest distance between a line 111, which connects a center 21 of the imaging unit 101R and a center 22 of the imaging unit 101L, and a center 23 of the channel 102, is denoted by yd, and the shortest distances between the line perpendicular from the center 23 to the line 111, and the centers 21 and 22, are respectively denoted by xd1 and xd2. FIG. 2B is a schematic diagram illustrating a state where the treatment instrument 24 is delivered from the channel by a length L, which is seen from the direction of the arrow 112 of FIG. 2A. FIG. 2C illustrates an image which is acquired in the imaging unit 101R at this time. The front end of the treatment instrument is located at a position 29 in the image, and the position 29 has pixel positions n and m when seen from a center pixel 28 of the image.

For simple description, the camera is assumed as a pinhole camera, and the thickness of the treatment instrument is disregarded. Further, the distance up to the image plane from the camera is denoted by f, the pixel pitch of the imaging plane is denoted by p, and the position of the front end on the image plane when the treatment instrument 24 is delivered by L (the distance of the image plane from the center 28) is denoted by y. At this time, the following relation is obtained.

$$n = \frac{y}{p} = \frac{f \cdot y d}{p \cdot L} \qquad [ Expression 1 ]$$

In the same way,

$$m = \frac{f \cdot xd 1}{p \cdot L} \qquad [ Expression 2 ]$$

In these parameters, p and f may be obtained in advance for each imaging condition. yd and xd1 become constant values by the stereoscopic endoscope to be used. Therefore, n and m may be obtained as a function of the length L in which the treatment instrument 24 is delivered. Consequently, when the template of the image of the treatment instrument is created in advance and the template matching is performed on the trace of (m, n) depicted by (Expression 1) and (Expression 2), the front end position can be easily detected.

This process is not limited only to the image of the imaging unit 101R, and for example, when the same detection is also performed on the image of 101L and the combination thereof is performed, the precision may be further improved.

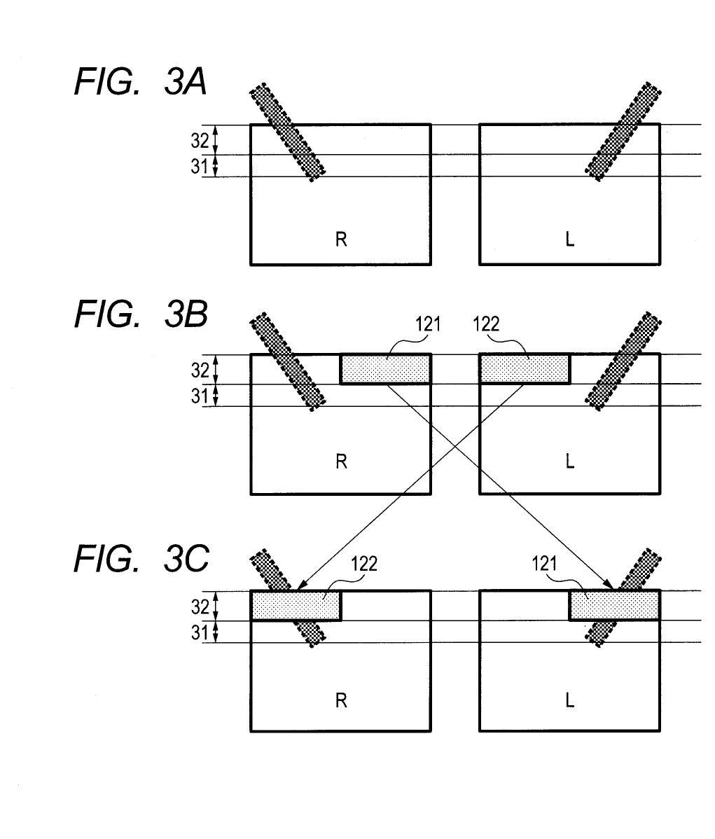

The process of the image processing unit 13 will be described by referring to FIGS. 3A, 3B, and 3C. An area 31 is an area to be displayed as a three-dimensional image, and an area 32 is an area to be displayed as a two-dimensional image.

In the image processing unit 13, the area 31 which is present within a predetermined distance in the front end of the treatment instrument and which is obtained by the area determining unit is specified. Further, the area 32 which is distant from the front end of the treatment instrument by a predetermined distance is specified. Note that, however, the area 32 may be determined as an area where only the treatment instrument is present.

The length of the predetermined distance used when specifying the area 31 is not particularly limited, and may be arbitrarily determined in consideration of a degree in which the visual interference is reduced. Although there is no particular limitation, Ny/4 pixels or less is desirable, where Ny denotes an image size (a number of pixels) in the longitudinal direction in FIG. 3A, for example. Further, since the position where the treatment instrument is first seen in the image is uniquely determined, the area 32 may be determined only in a direction in which the treatment instrument is first seen in the image when seen from the front end of the treatment instrument.

When the area of the treatment instrument seen in the image is smaller than the predetermined distance from the front end thereof, the area is not displayed as a two-dimensional image.

FIG. 3A illustrates images which are acquired by the imaging units 101R and 101L, and the area 32 will be displayed as a two-dimensional image. The method of displaying the area as a two-dimensional image is not particularly limited. For example, as illustrated in FIGS. 3B and 3C, an area corresponding to a portion where the treatment instrument is seen can be copied from the other image and overlapped.

That is, a corresponding area 122 within the image L is copied to the area where the treatment instrument is seen in the area 32 of the image R of FIG. 3B, so that an overlapped image is obtained as illustrated in FIG. 3C. Similarly in the image L, a corresponding area 121 within the image R is copied to the area 32 where the treatment instrument is seen in the area 32 of the image L of FIG. 3B, so that an overlapped image is obtained as illustrated in FIG. 3C. In this method, although the two-dimensional image of which the left and right positions are replaced with each other is displayed, since the treatment instrument is not seen in the areas which are copied from the image R and the image L as illustrated in FIG. 3B, no wobbling overlap of the treatment instrument is generated in the stereoscopic image. For this reason, the visual interference is reduced, and hence there is a merit that the feeling of fatigue can be lowered. Although a discontinuous portion is generated at the boundary due to the replacement of the image, this is not a considerable problem because the portion is not an area where the treatment instrument is operated.

Alternatively, the treatment instrument may be made to be invisible by covering the image of the area 32 with a mask or the like or interpolating an image around the area 32 instead of the process of replacing the area 32. In this case, the images of the areas 121 and 122 in FIG. 3C are obtained by the interpolation.

The image is displayed in the display unit 14. As the display unit 14, a conventional stereoscopic display device may be used.

As described above, it is possible to provide a stereoscopic endoscope image capable of reducing the visual interference by detecting the front end of the treatment instrument and the area which is seen in the image and replacing the area of the treatment instrument within the image other than a predetermined area of the front end with the area at the same position of the other image.

The front end of the treatment instrument may be detected by a method of using an image processing or by a method of installing a sensor at the front end thereof. In the case of using the image processing, a front end of a moving subject may be determined as the front end of the treatment instrument based on a change in the image, or the shape of the front end of the treatment instrument may be searched.

### Second Embodiment

In embodiment, the same effect is aimed by deforming the image, instead of replacing the area of a part of the treatment instrument seen in the image with the area at the same position of the other image in the first embodiment.

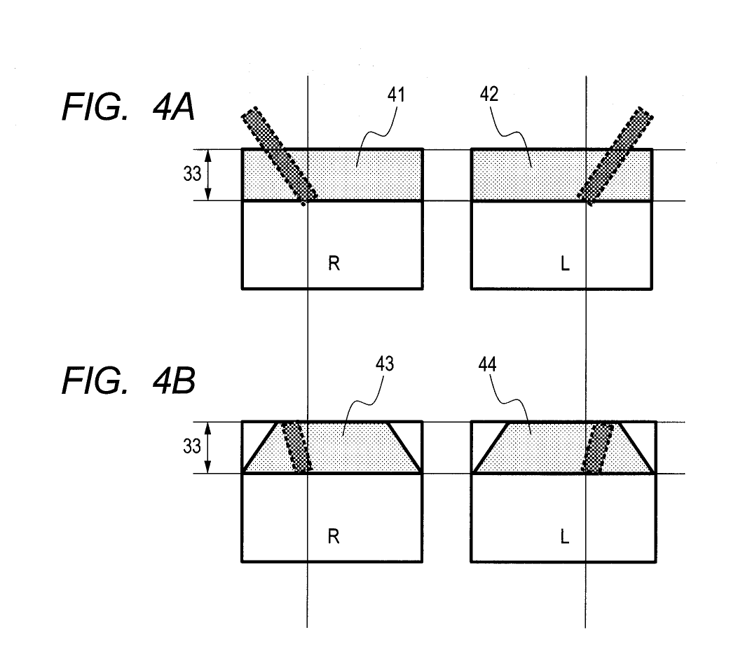

Referring to FIGS. 4A and 4B, in areas such as the rectangular areas 41 and 42 which are not easily displayed as a stereoscopic image, the disparity of the images is large. When a disparity is reduced in an area with a large disparity, that is, an area closer to the imaging unit 101, the stereoscopic image may be easily obtained. Therefore, for example, when the rectangular area 41 is mapped to the trapezoid area 43 and the rectangular area 42 is mapped to the trapezoid area 44, the disparity between the left and right images may be reduced. In this case, the treatment equipments appear as curved lines with an amount of thickness. In FIG. 4B, however, in order to simplify the explanation, they are drawn as straight lines with an amount of thickness. That is, in the embodiment, the apparatus includes a deformation area determining unit which sets an area where an image is deformed and a deformation unit which deforms the set area. This deformation may be realized by texture mapping, which is a general technique in a computer graphic. Although the end of the image is removed due to the trapezoid deformation, for example, black pixels representing that there is no image in the area can be inserted into the portion. The color of the inserted pixel is not limited to black, and may be any color.

Due to the above-described process, although a space different from the real state is perceived in the stereoscopic space, the visual interference which prohibits the stereoscopic image may instead be reduced. Since this portion is not an area where the treatment instrument is operated, as described above, an amount of deformation can be allowed in the area.

The front end of the treatment instrument within the image may be obtained by the method of the first embodiment. Alternatively, since an area closer to the imaging unit 101 (the area where the image is deformed) corresponds to an area which includes a place where the treatment instrument is first seen in the image, the area can be set to an area which includes the front end of the treatment instrument within the image and an arbitrary range from the front end in a direction toward a place where the treatment instrument is first seen in the image. The deformation of the rectangular area mapping shape is not limited to the above trapezoid provided that the deformation reduces the disparity.

### Other Embodiments

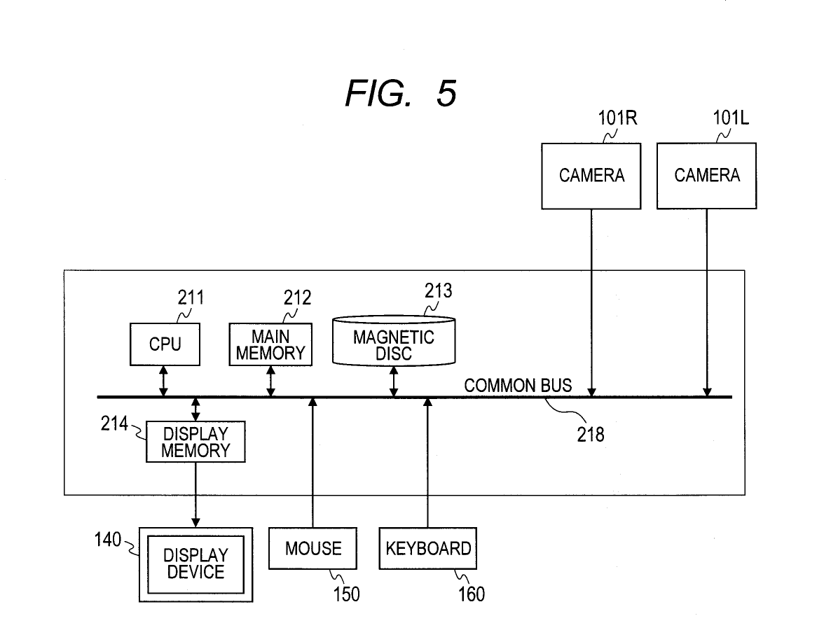

The first embodiment and the second embodiment can both be realized by a computer. FIG. 5 illustrates the configuration thereof. In the drawing, there are provided a CPU 211, a main memory 212, a magnetic disc 213, a display memory 214, a common bus 218, a display device 140, a mouse 150, and a keyboard 160. Then, the imaging units 101R and 101L of the stereoscopic endoscope are provided.

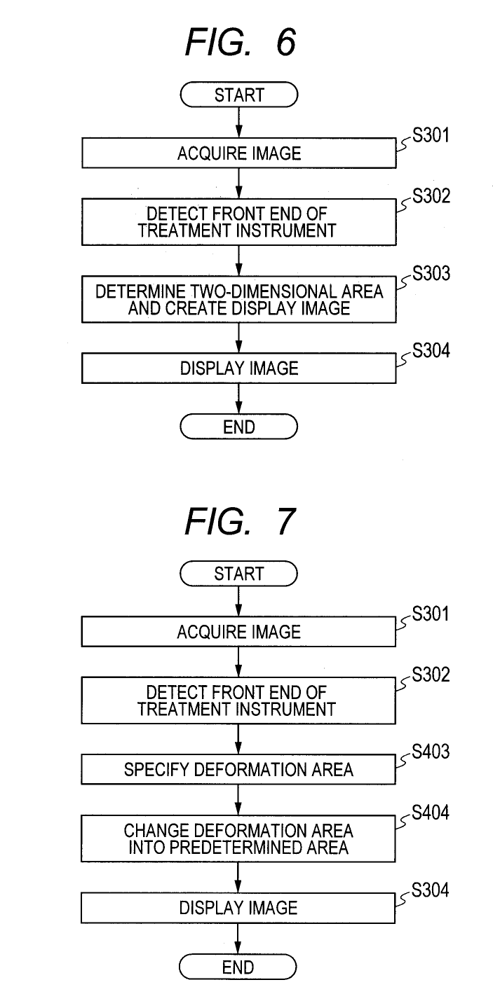

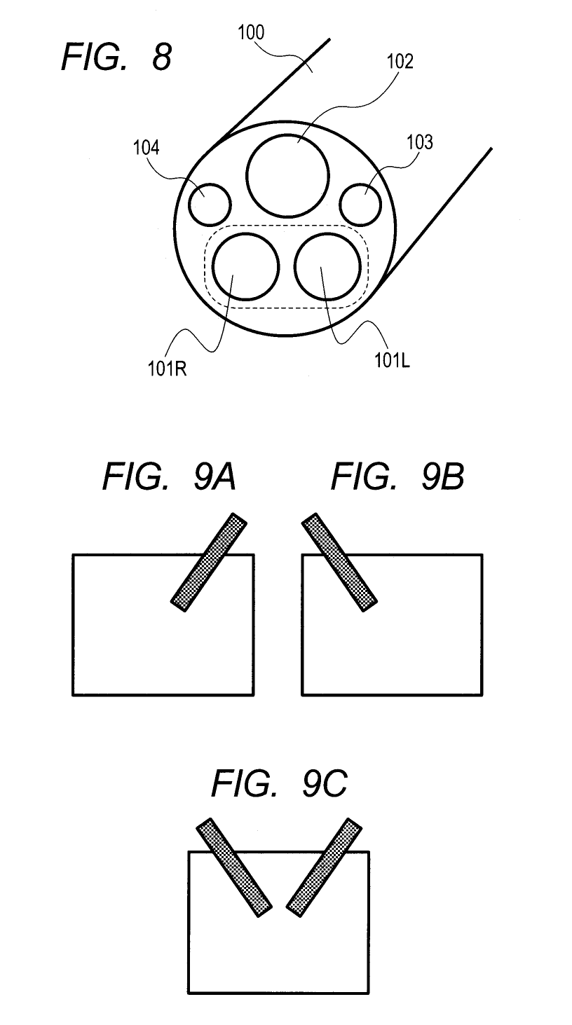

Referring to FIG. 6, first, in step S301, the images corresponding to the left and right eyes are acquired. In step S302, the front end of the treatment instrument which is seen in the image is detected. In step S303, the area of the treatment instrument distant by a predetermined distance or more from the front end detected in step S302 is detected, and the area is displayed as a two-dimensional image. The method of displaying a two-dimensional image is the same as that of the first embodiment. Subsequently, the obtained image is displayed in step S304.

Referring to FIG. 7, in the process of the second embodiment, S301, S302, and S304 are the same as those of the first embodiment. In step S403, the area which includes the front end of the treatment instrument is determined as the deformation area. Then, in step S404, the deformation area is deformed (mapped) as the predetermined area. In the case of the second embodiment, the area which includes the front end of the treatment instrument includes the rectangular areas 41 and 42, and the predetermined area includes the trapezoid areas 43 and 44.

As other methods, a method may be exemplified in which a low-pass filter, for example, a Gaussian filter, is applied to an area which is distant by a predetermined distance from the front end of the treatment instrument within the image. In this method, since blur occurs in an area where the filter is applied, the visual interference may be reduced.

With regard to the method of detecting the front end of the treatment instrument, a matching method is supposed in consideration of the case where the treatment instrument deforms or freely moves instead of the template matching on the trace of the treatment instrument described by referring to FIG. 2. As an example thereof, a method is supposed in which a sensor is installed in an endoscope body so as to detect an extent where a treatment instrument is delivered from a channel and the template matching searching range is determined based on the information thereof. In a method without using a sensor, since the place where the treatment instrument is first seen in the image is uniquely determined, a method may be adopted in which the place is monitored and the found portion is searched.

As described above, in the stereoscopic endoscope image, since a part of the area where the treatment instrument is seen is switched between the left and right images, visual interference can be reduced. Further, since the position where the treatment instrument is present may be checked by marking a part of the area of the front end of the treatment instrument as it is at all times, it is possible to prevent the body tissue from being damaged depending on a state where the treatment instrument is not visible or is not easily visible. Furthermore, when the treatment instrument seeing method is changed by partly deforming a space which is perceived as a stereoscopic display instead of the two-dimensional image, also, the visual interference may be reduced. Further, even in a method of adopting the image process in which the center and the periphery of the image are weighted differently, the visual interference may be reduced.

In the above-described embodiments, the invention has been described by exemplifying the endoscope with two imaging units. However, the stereoscopic observation may be performed in a manner such that a plurality of (two or more) imaging units are provided and the process of the invention is performed on any one of the plurality of images obtained in the respective imaging units. At this time, the process of the invention does not need to be performed on all images acquired in the plurality of imaging units, and the area where the two-dimensional image is displayed or the area where the image is deformed may be determined in only at least one arbitrary image from the obtained images.

Further, the imaging unit may include an optical system. One imaging optical system may include two or more imaging units. For example, a configuration with two or more imaging elements through one lens may be adopted.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2011-095206, filed Apr. 21, 2011, which is hereby incorporated by reference herein in its entirety.

What is claimed is:

1. A stereoscopic endoscope apparatus that includes a plurality of imaging units and a channel having an operable treatment instrument inserted therethrough and stereoscopically displays an image obtained from the plurality of imaging units, the stereoscopic endoscope apparatus comprising:
a front end detecting unit configured to detect a front end of the treatment instrument within at least one arbitrary image from the plurality of images acquired by the imaging units;
an area determining unit configured to determine an area to be displayed as a two-dimensional image; and
a two-dimensional image processing unit configured to two-dimensionally image the area to be displayed as a two-dimensional image,
wherein the area to be displayed as a two-dimensional image is an area where the treatment instrument is seen.

2. The stereoscopic endoscope apparatus according to claim 1,
wherein the area to be displayed as a two-dimensional image does not include the front end of the treatment instrument.

3. The stereoscopic endoscope apparatus according to claim 1,
wherein an area within a predetermined distance from the front end of the treatment instrument is not two-dimensionally imaged.

4. A stereoscopic endoscope apparatus that includes a plurality of imaging units and a channel having an operable treatment instrument inserted therethrough and stereoscopically displays an image obtained from the plurality of imaging units, the stereoscopic endoscope apparatus comprising:
a front end detecting unit configured to detect a front end of the treatment instrument within at least one arbitrary image from the plurality of images acquired by the imaging units;
a deformation area determining unit configured to determine an image deformation area within the image; and
an image deformation processing unit configured to deform an image within the deformation area, wherein the image deformation processing unit is an image processing unit configured to perform an image deformation for reducing a disparity of an image acquired by the plurality of images.

5. The stereoscopic endoscope apparatus according to claim 4,
wherein the deformation area is an area where the front end of the treatment instrument is not included and the treatment instrument is seen.

6. The stereoscopic endoscope apparatus according to claim 4,
wherein a shape of the deformed deformation area is a trapezoid.

7. An image processing method in a stereoscopic endoscope system that includes a plurality of imaging units and a treatment instrument and stereoscopically displays an image obtained from the plurality of imaging units, the image processing method comprising:
capturing an image;
detecting a front end of the treatment instrument within at least one arbitrary image from the plurality of images acquired by the imaging units;
determining an area where the front end of the treatment instrument is not included and the treatment instrument is seen; and
performing an image process of displaying the area of the image used for the stereoscopic display in the plurality of images as a two-dimensional image.

8. An image processing method in a stereoscopic endoscope system that includes a plurality of imaging units and a treatment instrument and stereoscopically displays an image obtained from the plurality of imaging units, the image processing method comprising:
capturing an image;
detecting a front end of the treatment instrument within at least one arbitrary image from the plurality of images acquired by the imaging units;
determining a deformation area that is an area distant from the front end of the treatment instrument by a predetermined distance and in which the treatment instrument is seen and the image is deformed; and
deforming the deformation area in a predetermined shape.
